(19) United States
(12) Patent Application Publication
(10) Pub. No.: US 2021/0169977 A1
(43) Pub. Date: Jun. 10, 2021

(54) Compositions of Transforming Growth Factor-Beta Type III Receptor and Uses for Ossification

(71) Applicant: Emory University, Atlanta, GA (US)

(72) Inventor: Steven Goudy, Decatur, GA (US)

(21) Appl. No.: 16/972,800

(22) PCT Filed: Jun. 6, 2019
(86) PCT No.: PCT/US2019/035779

§ 371 Date: Dec. 7, 2020

Related U.S. Application Data
(60) Provisional application No. 62/681,463, filed on Jun. 6, 2018.

(51) Int. Cl. A61K 38/17; A61K 38/39; A61L 27/18; A61L 27/52; A61L 27/54

(52) U.S. Cl. CPC A61K 38/179; A61K 38/39; A61L 27/18; A61L 2430/02; A61L 27/54; A61L 2300/252; A61L 2400/06; A61L 27/52

(57) ABSTRACT

This disclosure relates to compositions containing transforming growth factor-beta type III receptor (TβRIII) for ossification and methods related thereto. In certain embodiments, this disclosure relates to methods of using these compositions to improve bone formation after surgery to repair a bone void such as an orofacial cleft, cleft palate, or cleft lip. In a typical embodiment, the bone graft composition comprises exogenously added transforming growth factor-beta type III receptor (TβRIII) or variants and/or TβRII expressing cells. In certain embodiments, the graft composition is a biodegradable hydrogel based polymer impregnated with TβRIII or variants and/or TβRIII expressing cells.

### CROSS-REFERENCE TO RELATED APPLICATIONS

This application claims the benefit of U.S. Provisional Application No. 62/681,463 filed Jun. 6, 2018. The entirety of this application is hereby incorporated by reference for all purposes.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 17006PCT_ST25.txt. The text file is 36 KB, was created on Jun. 6, 2019, and is being submitted electronically via EFSWeb.

### BACKGROUND

If bones and tissues of the face do not join properly during fetal growth, a cleft palate, cleft lip, or both, can develop. Surgical interventions are commonly used to correct these defects. Autogenous bone grafting from the iliac crest is preferred for addressing large gaps in the bone structure. However, the procedure often results in complications such as arterial injury, hernia, chronic pain, nerve injury, and infection.

An alternative bone regenerative approach utilizes bone morphogenetic protein 2 (BMP-2) absorbed into a collagen sponge. Recombinant human BMP-2 has been approved by the FDA in synthetic bone grafts such as INFUSE™. BMP-2 induces local progenitor cells to form new bone; however, it sometimes results in significant and undesirable inflammatory responses. The FDA label for INFUSE™ indicates that is should not be used in in patient who are less than 18 years of age. Thus, there is a need for improved grafting materials to surgically correct orofacial clefts in young patients.

Transforming growth factor-beta (TGF-β) and bone morphogenic protein (BMP) are important in both skeletal development and bone homeostasis. TGF-β and BMP receptor signaling are implicated in osteoblast, skeletal development, and bone formation. See Wu et al. Bone Research (2016) 4, 16009. Transforming growth factor-beta type III receptor (TβRIII, also known as betaglycan) is thought to be involved in cancer, vascular, and osteoblast development. See Hill et al. Dev Dyn. 2015, 244(2):122-33, see Gatza et al. Cell Signal. 2010, 22(8): 1163-1174. Human reference sequence of TβRIII is GenBank accession number CAB64374.1. See also U.S. Pat. No. 6,010,872 which reports recombinant TβRIII polypeptides.

References cited herein are not an admission of prior art.

### SUMMARY

This disclosure relates to compositions containing transforming growth factor-beta type III receptor (TβRIII) for ossification and methods related thereto. In certain embodiments, this disclosure relates to methods of using these compositions to improve bone formation after surgery to repair a bone void such as an orofacial cleft, cleft palate, or cleft lip. In a typical embodiment, the bone graft composition comprises exogenously added transforming growth factor-beta type III receptor (TβRIII) or variants and/or TβRIII expressing cells. In certain embodiments, the graft composition is a biodegradable hydrogel based polymer impregnated with TβRIII or variants and/or TβRIII expressing cells.

In certain embodiments, this disclosure contemplates using TβRIII or variants, soluble TβRIII, and/or TβRIII expressing cells, e.g., cell genetically modified to express TβRIII on the cell membrane at greater concentration than found in normal or wild-type cells, to induce osteoblast commitment providing an osteoinductive scaffold for bone grafting.

In certain embodiments, TβRIII comprises SEQ ID NOs: 1, 4, 5, or amino acids Gly21-Asp781 of SEQ ID NO: 1 or variant thereof. In certain embodiments, TβRIII expressed on a cell or derived from TβRIII overexpressed on a cell. In certain embodiments, the graft comprises polyethylene glycol, polyethylene glycol methyl ether maleimide polymer, collagen, or other hydrogel matrix. In certain embodiments, the disclosure contemplates TβRIII, variants, and/or TβRIII expressing cells in a hydrogel scaffolding produced from polyethylene glycol maleimide (PEG-MAL) macromers.

In certain embodiments, the graft composition comprises TβRIII or variants, soluble TβRIII, and/or TβRIII expressing cells and other osteogenic material such as calcium phosphates and/or bone granules, hydroxyapatite and/or beta-tricalcium phosphate, alpha-tricalcium phosphate, polysaccharides or combinations thereof. In some embodiments, crushed bone granules, typically obtained from the subject, are optionally added to the graft composition. In some embodiments, the graft composition comprises bioglass and/or calcium sulphate. In some embodiments, the graft composition comprises hydroxyapatite and tricalcium phosphate.

In some embodiments, the disclosure relates to bone graft compositions comprising a TβRIII, variants, soluble TβRIII, and/or TβRIII expressing cells disclosed herein and a graft matrix. Typically, the matrix is a hydrogel or other hydrophilic polymer which is biodegradable. In other embodiments, the matrix comprises a collagen sponge and/or a compression resistant type I collagen and optionally calcium phosphates.

In some embodiments, the graft comprises TβRIII, variants, soluble TβRIII, and/or TβRIII expressing cells and contains osteogenic material can be obtained from autogenic or allogenic sources and includes, autograft bone, bone of the iliac crest, teeth, autogenic bone marrow aspirate, autogenic lipoaspirate, allogenic cadaveric bone, allogenic bone marrow aspirate, allogenic lipoaspirate, and blends and mixtures thereof.

In certain embodiments, the disclosure contemplates a graft composition or matrix comprising TβRIII, variants, soluble TβRIII, and/or TβRIII expressing cells as disclosed herein wherein the graft matrix is a collagen or demineralized bone matrix or ceramic or other scaffold disclosed herein with or without exogenous cells.

In certain embodiments, bone graft compositions comprise TβRIII, variants, soluble TβRIII, and/or TβRIII expressing cells disclosed herein and optionally a bone morphogenetic protein such as BMP-2, BMP-5, or BMP-7 and/or optionally another growth factor. In some embodiments, the disclosure relates to kits comprising TβRIII, variants, soluble

TβRIII, and/or TβRIII expressing cells as disclosed herein and a graft composition and/or graft matrix. In certain embodiments, the kits further optionally comprise a transfer device, such as a syringe or pipette. In certain embodiments, the kits further optionally comprise a bone morphogenetic protein and/or another growth factor.

In some embodiments, the disclosure relates to methods of generating osteoblasts comprising mixing or administering an effective amount of TβRIII, variants, soluble TβRIII, and/or TβRIII expressing cells as disclosed herein into or with cells capable of osteoblastic differentiation, such as mesenchymal stem cells and pre-osteoblastic cells.

In some embodiments, the disclosure relates to methods of forming bone comprising implanting a graft composition or matrix comprising TβRIII, variants, soluble TβRIII, and/or TβRIII expressing cells as disclosed herein in a subject under conditions such that bone forms in or around the graft. Typically, the subject has a void in the bony structure wherein the graft composition is implanted in the void. In certain embodiments, the void is in a bone that is the result of an orofacial cleft, a cleft palate, cleft lip, or the void is in an extremity, maxilla, mandible, pelvis, spine and/or cranium. In certain embodiments, the void is a result of surgical removal of bone. In certain embodiments, the void is in a bone of the face or cranium. In certain embodiments, the void is between bone and an implanted medical device.

In another embodiment, the method further comprises the step of securing movement of bone structure with a fixation system, and removing the system after bone forms in the implanted graft. In certain embodiments, the disclosure contemplates regional bone enhancement for osteopenic bones before they fracture (e.g. hip, vertebral body, etc.) by deliver locally to induce local bone formation.

In certain embodiments, the disclosure relates to methods of growing bone in subject by locally administering, such as by injection, a composition comprising TβRIII, variants, soluble TβRIII, and/or TβRIII expressing cells as disclosed herein, optionally in combination with a growth factor, about the area of desired bone growth. In certain embodiments, the disclosure relates to methods of growing bone comprising administering a pharmaceutical composition comprising TβRIII, variants, soluble TβRIII, and/or TβRIII expressing cells as disclosed herein, or pharmaceutically acceptable salts thereof to a subject in an area of desired growth, wherein the administration is localized directly about the area of desired growth. In certain embodiments, the administration is oral or is not oral administration. In certain embodiments, the administration is through a catheter or hypodermic needle with a tip that is not in a vein. In certain embodiments, the administration is by injection into the subcutaneous tissue or in or about an area typically occupied by bone.

In certain embodiments, the method contemplates implanting a graft composition in a desired area of the subject and locally administering a composition comprising a TβRIII, variants, soluble TβRIII, and/or TβRIII expressing cells as disclosed herein, optionally in combination with a growth factor, in the graft or about the area of the graft implant such as by injection.

In some embodiments, the disclosure relates to pharmaceutical compositions comprising TβRIII, variants, soluble TβRIII, and/or TβRIII expressing cells as disclosed herein or pharmaceutically acceptable salts thereof.

In some embodiments, the disclosure relates to methods of preventing or treating a bone degenerative disease, comprising administering a pharmaceutical composition comprising TβRIII, variants, soluble TβRIII, and/or TβRIII expressing cells as disclosed herein or a pharmaceutically acceptable salts thereof, to a subject at risk for, exhibiting symptoms of, or diagnosed with a bone degenerative disease. In certain embodiments, the subject is diagnosed with a genetic profile that correlates with or indicates an increased risk of forming abnormal bone structures such as an orofacial cleft, a cleft palate, cleft lip, or combinations thereof.

In certain embodiments, the administration is systemic, or administration is achieved through oral delivery, intravenous delivery, parenteral delivery, intradermal delivery, percutaneous delivery, or subcutaneous delivery. In some embodiments, the disease is osteoporosis, osteitis deformans, bone metastasis, multiple myeloma, primary hyperparathyroidism, or osteogenesis imperfecta.

In some embodiments, the disclosure relates to methods for decreasing the time required to form new bone in the presence of a bone morphogenetic protein, comprising co-administering TβRIII, variants, soluble TβRIII, and/or TβRIII expressing cells as disclosed herein and another active ingredient.

In some embodiments, the disclosure relates to a process for engineering bone tissue comprising combining TβRIII, variants, soluble TβRIII, and/or TβRIII expressing cells as disclosed herein with a cell selected from the group consisting of osteogenic cells, pluripotent stem cells, mesenchymal cells, and embryonic stem cells.

In certain embodiments, the disclosure relates to using TβRIII, variants, soluble TβRIII, and/or TβRIII expressing cells as disclosed herein in the production of a medicament for the treatment or prevention of a bone disease or other applications disclosed herein.

In certain embodiments, the disclosure contemplates delivery of compositions comprising compounds disclosed herein via a liquid or flowable gel optionally including collagen, hydroxyapatite, demineralized bone, or polymer matrix or others, e.g., as disclosed herein. In certain embodiments, the compositions are injected into the central cavity or interstices of a structural element such as a bone cage made from allograft, polymer, metal such as titanium or aluminum, polyether ether ketone (PEEK) such as those generated from 4,4′-difluorobenzophenone, or other polymer.

### BRIEF DESCRIPTION OF THE DRAWINGS

FIG. 1 shows data for palate mesenchymal cells that were TβRIII−/− which were unable to mineralize to form bone in osteogenic media (OM).

FIG. 2A shows a picture of TβRIII−/− palate mesenchymal cells indicating they were unable to mineralize to form bone in osteogenic media (OM).

FIG. 2B shows a picture indicating the rescue of the TβRIII−/− palate mesenchymal cells using TβRIII-FL adenovirus infection with normal mineralization.

FIG. 3 shows data indicating overexpression of TβRIII using adenoviral vector in HEPM cells with osteogenic media induces mineralization.

FIG. 4 shows data when HEPM cells in growth media responded to soluble TβRII therapy with induction of alkaline phosphatase activity at 12.5, 25 and 50 ng/ml.

FIG. 5 illustrates a sequence comparison (81% identity) of Homo sapiens TGF-beta type III receptor NP_003234.2 (SEQ ID NO:1) and Mus musculus TGF-beta type III receptor NP_035708.2 (SEQ ID NO: 2).

FIG. 6 illustrates a sequence comparison (81% identity) of Homo sapiens TGF-beta type III receptor NP_003234.2 (SEQ ID NO:1) and Rattus norvegicus TGF-beta type III receptor NP_058952.1 (SEQ ID NO: 3).

FIG. 7A shows data from micro-CT images of cranial defect in mice. BMP2 and soluble TGF-beta type III receptor incorporated in hydrogel were place in the defect area. Bone formation was then analyzed after 12 weeks. Bone development was characterized by quantification of the bone volume.

FIG. 7B shows immunohistochemistry images of mouse defect treated with TGF-beta type III receptor incorporated in a hydrogel. H&E images of negative controls (defect alone and hydrogel).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. “Ossification” refers to the process of laying down new bone by cells called osteoblasts.

The term includes the growth in healing bone fractures treated by cast or by open reduction and stabilization by metal plate and screws. Ossification may also result in the formation of bone tissue in an extra-skeletal location.

The term “bone graft composition” refers to materials that are substantially physiologically compatible when residing in bone area, void, or exterior site. In certain embodiments, the bone graft composition may be a bone graft matrix such as a collagen sponge or a mixture of polymers and salts.

The terms “exogenously added” in reference to a component of a mixture refers to a component that does not naturally originate specifically from one of the other components in a mixture. This should not be taken to mean that exogenously added component may not be of natural origin. For example, tissue may produce a polypeptide and a dehydrated tissue may contain the polypeptide. However, dehydrated tissue comprising an exogenously added polypeptide is not referring to the polypeptide that was produced by the tissue or a polypeptide that exists in the tissue component after dehydration. An exogenously added polypeptide may be synthesized, isolated, or purified external from a different sample of the same tissue which is then added to the tissue or dehydrated tissue component.

As used herein, the term “biodegradable” refers to a material that when transplanted into an area of a subject, e.g., human, will be degraded my biological mechanism such that the material will not persist in the area for over a long period of time, e.g., material will be removed by the body after a couple weeks or months. In certain embodiments, the disclose contemplates that the biodegradable material will not be found at the transplanted location after six months, a year, or two years.

As used herein, “subject” refers to any animal, preferably a human patient, livestock, or domestic pet. As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, the term “calcium phosphate(s)” refers to minerals containing calcium ions together with orthophosphates, metaphosphates or pyrophosphates and optionally hydroxide ions. Tricalcium phosphate is a calcium phosphate with formula Ca₃(PO₄)₂. The common mineral apatite has the basic formula Ca₅(PO₄)₃X, where X is a ion, typically a halogen or hydroxide ion, or a mixture. Hydroxyapatite refers to apatite where X is mainly hydroxide ion.

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG (SEQ ID NO: 8) and GGGGT (SEQ ID NO: 9) have a sequence identity of 4 out of 5 or 80%. For example, the polypeptides GGGPPP (SEQ ID NO: 10) and GGGAPPP (SEQ ID NO: 11) have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic-A V I L; Charged positive: R K H; Charged negative—D E; Polar—S T N Q. These amino acid groups are also considered to be conserved substitutions.

### Graft Compositions

In some embodiments, the disclosure relates to graft compositions comprising Transforming growth factor-beta type III receptor (TβRIII, also known as betaglycan) and optionally other growth factor(s). In certain embodiments, the graft may be a porous or non-porous material that comprises or is coated with a compositions disclosed herein, e.g., a hydrogel.

In certain embodiments, a hydrogel may contain polyethylene glycol or polyethylene glycol macromers or dendrimers terminally substituted with reactive coupling groups, e.g., PEG-diacrylate, 4-arm PEG-maleimide (PEG-4MAL) macromere, 4-arm PEG-acrylate (PEG-4A), 4-arm PEG-vinylsulfone (PEG-4VS). The macromers or dendrimers may be further functionalized with crosslinking agents or polypeptides with desirable functional attributes, e.g., the graft or hydrogel may be designed to contain an adhesive RGD sequence or a protease-cleavable peptide sequence to facilitate the biodegradable nature of an implanted composition.

In certain embodiments, these compositions may be created from polymers, demineralized bone matrix (DBM), bone granules, and ceramics such as calcium phosphates (e.g. hydroxyapatite and tricalcium phosphate), bioglass, and calcium sulphate. In certain embodiments, it is contemplated that the bone granules are autogenous, i.e., derived from the subject that is to receive the implanted bone graft. In certain embodiments, bone granules or demineralized (decalcified) bone matrix (DBM) are allogeneic, i.e., derived from somewhere other than the subject such as from another human or other animal. The grafts may contain carrier-beds of collagen or biodegradable polymers, antibacterial agents, bone morphogenetic proteins, and growth factors (platelet-derived growth factor, insulin-like growth factor, vascular endothelial and fibroblast growth factors), and bone marrow aspirate.

Demineralized bone matrix (DBM) typically contains collagen (mostly type I with some types IV and X), non-collagenous proteins and growth factors, a variable percent of residual calcium phosphate mineral. DBM is typically derived from bone morsellized to defined particles or fibers and subjected to acid demineralization followed by one or more rounds of freeze-drying, e.g., the mineral phase is extracted from the particulate whole donor bone with hydrochloric acid, leaving the organic matrix intact. The demineralized bone powder can be formulated into putties, pastes, flexible, or pre-formed strips by integration with a carrier, e.g., polymer, collagen, albumin, carboxymethyl cellulose, lecithin, hydrogel, gelatin, cancellous chips, alginate salt.

In certain embodiments, the disclosure relates to graft compositions comprising TβRIII or variants which are covalently linked or are not covalently linked to bone graft compositions or scaffolds. In some embodiments, these compositions may be combined with growth factor(s).

Bioglass refers to materials of SiO₂, Na₂O, CaO and P₂O₅ in specific proportions. The proportions differ from the traditional soda-lime glasses in lower amounts of silica (typically less than 60 mol %), higher amounts of sodium and calcium, and higher calcium/phosphorus ratio. A high ratio of calcium to phosphorus promotes formation of apatite crystals; calcium and silica ions can act as crystallization nuclei. Some formulations bind to soft tissues and bone, some only to bone, some do not form a bond at all and after implantation get encapsulated with non-adhering fibrous tissue, and others are completely absorbed overtime. Mixtures of 35-60 mol % SiO₂, 10-50 mol % CaO, and 5-40 mol % Na₂O bond to bone and some formulations bond to soft tissues. Mixtures of >50 mol % SiO₂, <10 mol % CaO, <35 mol % Na₂O typically integrate within a month. Some CaO may be replaced with MgO and some Na₂O may be replaced with K₂O. Some CaO may be replaced with CaF₂.

In some embodiments, the disclosure relates to a graft composition comprising TβRIII and/or polysaccharides such as hyaluronate, alginate, cellulose or cellulose derivatives such as, but not limited to, hydroxypropyl cellulose, methyl cellulose, ethyl cellulose, and carboxymethyl cellulose. Typically, cellulose derivatives are used in graft compositions that produce a paste or putty.

In some embodiments, the disclosure relates to bone graft compositions comprising a bone morphogenetic protein and TβRIII and a graft matrix. The matrix is typically a polymer designed to hold bone compatible salts, such as calcium phosphates, for replacement during bone growth. An example is a bovine Type I collagen embedded with biphasic calcium phosphate granules. Optionally, matrix compositions may also include one or more agents that support the formation, development and growth of new bone, and/or the remodeling thereof. Typical examples of compounds that function in, such a supportive manner include extracellular matrix-associated bone proteins such as alkaline phosphatase, osteocalcin, bone sialoprotein (BSP) and osteocalcin, phosphoprotein (SPP)-1, type I collagen, fibronectin, osteonectin, thrombospondin, matrix-gla-protein (MGP), SPARC, and osteopontin.

In certain embodiments, the graft matrix can be made up of a hydrogel polymer. Typically, a hydrogel is made-up of polymers and copolymers substituted with an abundance of hydrophilic groups, such as polyethylene glycol and/or terminal hydroxyl or carboxyl groups. In certain embodiments, the graft composition is biodegradable. In certain embodiments, the matrix comprises homopolymers and copolymers consisting of glycolide and lactide. For certain embodiments, the graft composition comprises a matrix of hydroxyethylmethacrylate or hydroxymethylmethyacrylate polymers containing hydroxyapatite in a mineral content approximately that of human bone. Such a composition may also be made with crosslinkers comprising an ester, anhydride, orthoester, amide, or peptide bond. In some embodiments, crosslinkers contain the following polymers: polyethylene glycol (PEG), polylactic acid, polyglycolide or combinations thereof.

In certain embodiments, graft comprises recombinant human platelet-derived growth factor (becaplermin).

In certain embodiments, graft comprises an antimicrobial silver wound dressing, silver-coated synthetic mesh, e.g., a synthetic layer of nylon, coated with silver.

In certain embodiments, graft comprises platelet rich plasma (PRP), derived from the blood of a subject after high-speed centrifugation or autologous conditioned plasma (ACP), removal of white blood cells. The blood or platelet rich plasma portion may be activated with various reagents to convert the blood protein fibrinogen into fibrin. This fibrin-rich gel-like substance is then immediately applied to the graft.

In certain embodiments, graft comprises bone marrow aspirate, e.g. derived via needle aspiration of bone marrow. In certain embodiments, the bone graft comprises mesenchymal stem cells. In certain embodiments, the bone graft comprises silicate and calcium phosphate combined with autologous bone marrow aspirate (BMA). In certain embodiments, graft comprises blood mixed with microfibrillar collagen and thrombin.

In certain embodiments, the bone graft comprises beta tricalcium phosphate (β-TCP) combined with recombinant human platelet-derived growth factor BB (rhPDGF-BB). In certain embodiments, the bone graft comprises Type I bovine collagen and hydroxyapatite mixed with bone marrow aspirate.

In certain embodiments, the graft composition may contain one or more antibiotics and/or anti-inflammatory agents. Suitable antibiotics include, without limitation, nitroimidazole antibiotics, tetracyclines, penicillins, cephalosporins, carbapenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, ciclacillin, dicloxacillin, methicillin, oxacillin, piperacillin, ticarcillin, flucloxacillin, cefuroxime, cefetamet, cefteram, cefixime, cefoxitin, ceftazidime, ceftizoxime, latamoxef, cefoperazone, ceftriaxone, cefsulodin, cefotaxime, cephalexin, cefaclor, cefadroxil, cefalotin, cefazolin, cefpodoxime, ceftibuten, aztreonam, tigemonam, erythromycin, dirithromycin, roxithromycin, azithromycin, clarithromycin, clindamycin, lincomycin, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracycline, nitrofurantoin, nalidixic acid, gentamicin, rifampicin, amikacin, netilmicin, imipenem, cilastatin, chloramphenicol, furazolidone, nifuroxazide, sulfadiazine, sulfamethoxazole, bismuth sub salicylate, colloidal bismuth subcitrate, gramicidin, mecillinam, cloxiquine, chlorhexidine, dichlorobenzyl alcohol, methyl-2-pentylphenol or any combination thereof.

Suitable anti-inflammatory compounds include both steroidal and non-steroidal structures. Suitable non-limiting examples of steroidal anti-inflammatory compounds are corticosteroids such as hydrocortisone, cortisol, triamcinolone, alpha-methyl dexamethasone, dexamethasone-phosphate, beclomethasone dipropionates, clobetasol valerate, desonide, desoximetasone, desoxycorticosterone acetate, dexamethasone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, prednisone, paramethasone, prednisolone, prednisone, beclomethasone dipropionate, and triamcinolone. Mixtures of the above steroidal anti-inflammatory compounds may also be used.

Non-limiting examples of non-steroidal anti-inflammatory compounds include nabumetone, celecoxib, etodolac, nimesulide, gold, oxicams, such as piroxicam, isoxicam, meloxicam, tenoxicam, sudoxicam, the salicylates, such as aspirin, disalcid, benorylate, trilisate, safapryn, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

### Bone Grafting Methods

Bone grafting is possible because bone tissue, unlike most other tissues, has the ability to regenerate if provided the space into which to grow with appropriate chemical signals. With regard to synthetic grafts, as native bone grows, it typically replaces most or all of the artificial graft material, resulting in an integrated region of new bone. However, with regard to certain embodiments of the disclosure, it is not intended that new bone must remove all artificial material. In addition, with regard to certain embodiments of the disclosure, it is not intended that graft location need contact any other bone of the skeletal system.

In certain embodiments, the disclosure relates to a method of forming bone comprising implanting a graft composition comprising TβRIII or variants in a subject. In certain embodiments, the disclosure relates to methods of forming bone comprising implanting a graft composition comprising a bone morphogenetic protein and TβRIII or variants in a subject. The graft may be the result of a void created by surgical removal or created as a result of an attempt to correct a physical abnormality, such as but not limited to, cranial bones; frontal, parietal, temporal, occipital, sphenoid, ethmoid; facial bones; mandible, maxilla, palatine, zygomatic, nasal, lacrimal, vomer, inferior nasal conchae; shoulder girdle; scapula or shoulder blade, clavicle or collarbone; in the thorax; sternum, manubrium, gladiolus, and xiphoid process, ribs; in the vertebral column; cervical vertebrae, thoracic vertebrae; lumbar vertebrae; in the arms, humerus, radius, ulna; in the pelvis; coccyx; sacrum, hip bone (innominate bone or coxal bone); in the legs; femur, patella, tibia, and fibula. It is contemplated that the graft may be added for cosmetic purposes, e.g., cheek augmentation. In the case of a broken bone or removal of a bone during surgery, it may be desirable to secure movement of bone structure with a fixation system and remove the system after bone forms in the implanted graft.

With regard to prostheses, it may be desirable to grow bone between existing bone and an implanted device, or in preparation of an implanted device, such as in the case of a hip replacement, knee replacement, and dental implant, i.e., artificial tooth root used to support restorations that resemble a tooth or group of teeth.

In some embodiments, the disclosure relates to three-dimensional structures made of biocompatible and biodegradable bone graft materials in the shape of the bone infused with compositions disclosed herein to promote bone growth. Implants can be used to support a number of prostheses. A typical implant consists of a titanium device. In certain embodiments, the graft compositions disclosed herein contain implants.

With regard to a sinus augmentation or alveolar ridge augmentation, surgery may be performed as an outpatient under general anesthesia, oral conscious sedation, nitrous oxide sedation, intravenous sedation or under local anesthesia. Bone grafting is used in cases where there is a lack of adequate maxillary or mandibular bone in terms of depth or thickness. Sufficient bone is needed in three dimensions to securely integrate with the root-like implant. Improved bone height is important to assure ample anchorage of the root-like shape of the implant.

In a typical procedure, the clinician creates a large flap of the gingiva or gum to fully expose the bone at the graft site, performs one or several types of block and onlay grafts in and on existing bone, then installs a membrane designed to repel unwanted infection-causing bacteria. Then the mucosa is carefully sutured over the site. Together with a course of systemic antibiotics and topical antibacterial mouth rinses, the graft site is allowed to heal. The bone graft produces live vascular bone and is therefore suitable as a foundation for the dental implants.

In certain embodiments, the disclosure relates to methods of performing spinal fusion using TβRIII or variants disclosed herein. Typically, this procedure is used to eliminate the pain caused by abnormal motion of the vertebrae by immobilizing the vertebrae themselves. Spinal fusion is often done in the lumbar region of the spine, but the term is not intended to be limited to method of fusing lumbar vertebrae. Patients desiring spinal fusion may have neurological deficits or severe pain, which has not responded to conservative treatment. Conditions where spinal fusion may be considered include, but are not limited to, degenerative disc disease, spinal disc herniation, discogenic pain, spinal tumor, vertebral fracture, scoliosis, kyphosis (i.e, Scheuermann's disease), spondylolisthesis, or spondylosis.

In certain embodiments, different methods of lumbar spinal fusion may be used in conjunction with each other. In one method, one places the bone graft between the transverse processes in the back of the spine. These vertebrae are fixed in place with screws and/or wire through the pedicles of each vertebra attaching to a metal rod on each side of the vertebrae. In another method, one places the bone graft between the vertebrae in the area usually occupied by the intervertebral disc. In preparation for the spinal fusion, the disc is removed entirely. A device may be placed between the vertebra to maintain spine alignment and disc height. The intervertebral device may be made from either plastic or titanium or other suitable material. The fusion then occurs between the endplates of the vertebrae. Using both types of fusion is contemplated.

### Transforming Growth Factor-Beta (TGF-β) Type III

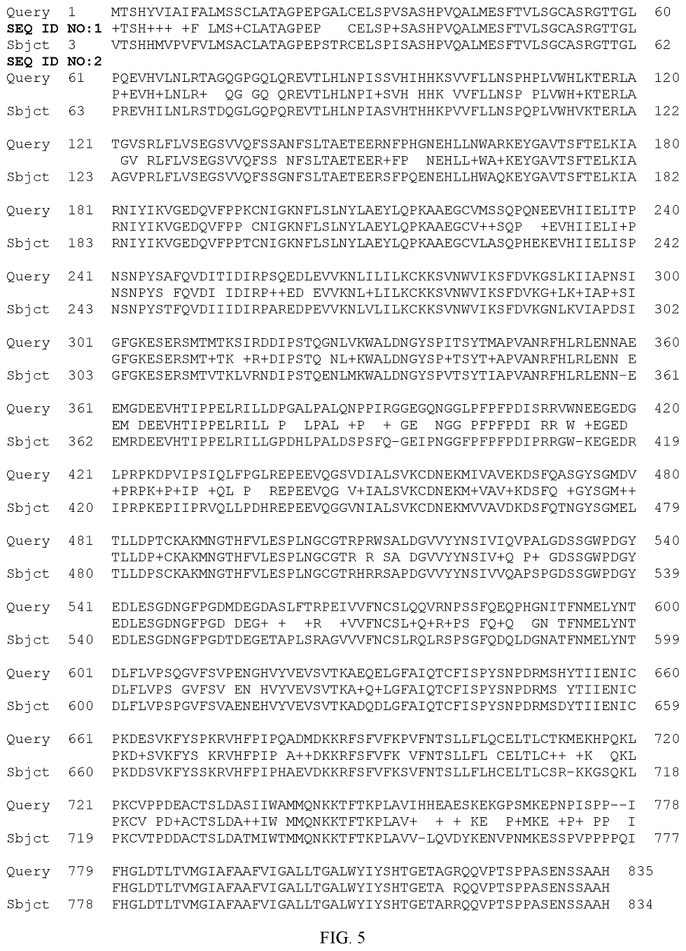

Human transforming growth factor-beta (TGF-β) type III (TβRIII, also known as betaglycan) is an 851 amino acid transmembrane proteoglycan, which contains a N-terminal 766 amino acid extracellular domain, a hydrophobic transmembrane domain, and a short 42 amino acid cytoplasmic domain. See FIG. 5, SEQ ID NO: 1. TβRIII binds and stabilizes TGFβ ligands and presents them to adjacent TGFβR 1 and 2, leading to intracellular activation of the SMAD pathway. Deletion of the cytoplasmic domain does not inhibit the ability of TβRIII to present ligand to adjacent TGFβ receptors. TβRIII can also undergo ectodomain shedding, releasing soluble TβRIII. Shedding of the ectodomain of TβRIII has been shown to inhibit TGFβ signaling where the soluble TβRIII sequesters TGFβ ligand. TβRIII mutants with impaired or enhanced ectodomain shedding are known. See Elderbroom et al. Mol Biol Cell. 2014, 25(16): 2320-2332. M742A mutants exhibits reduced ectodomain shedding. Mutations between amino acids 760-778 increase shedding.

In certain embodiments, this disclosure contemplates using full length TβRIII, variants, soluble TβRIII, and/or TβRIII expressing cells, e.g., cell genetically modified to express TβRIII on the cell membrane at greater concentration than found in normal or wild-type cells, to induce osteoblast commitment providing an osteoinductive scaffold for bone grafting.

Using a human embryonic palate fibroblast cell line, one can use adenoviral or lentiviral vectors to overexpress TβRIII full length (FL), variants, soluble and TβRIII with cytoplasmic domain or with a cytoplasmic domain deletion and intracellular deletion forms of TβRIII.

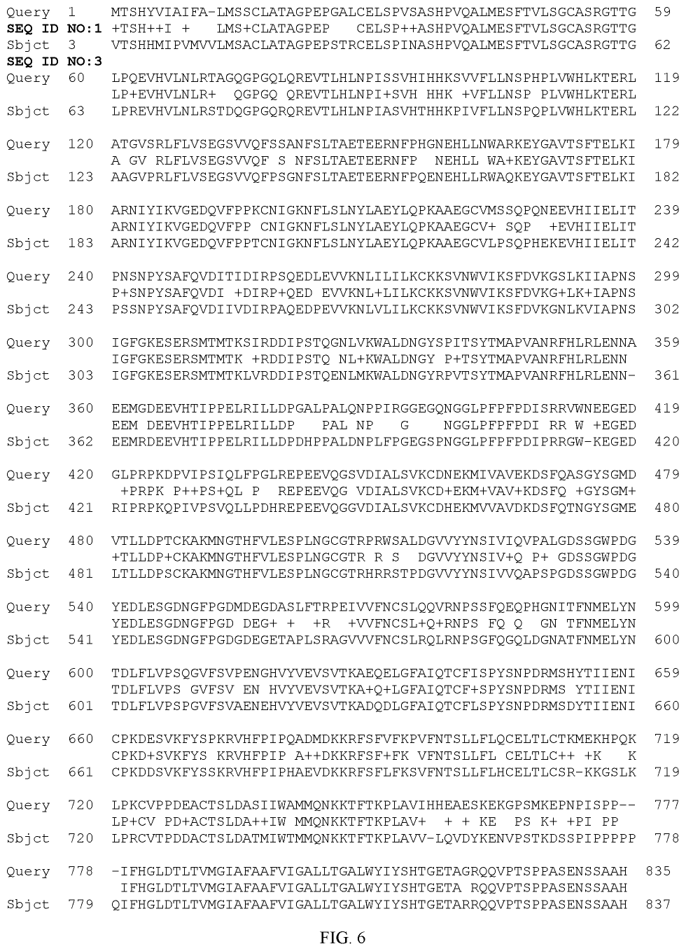

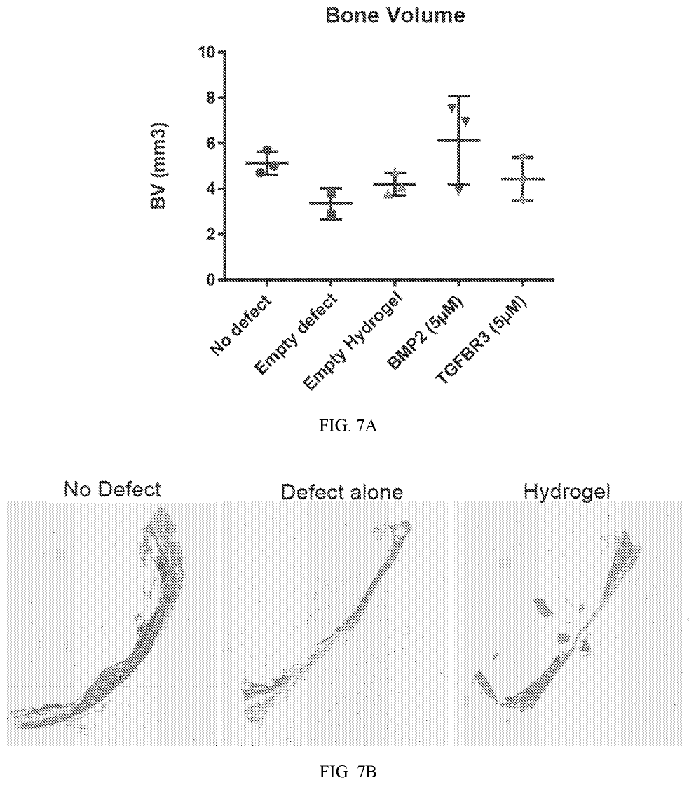

In certain embodiments, the disclosure contemplates that operable variants of TβRIII may contain the extracellular domain. In certain embodiments, the disclosure contemplates that operable variants of TβRIII may be derived from other species such as from the mouse (mus musculus, see human sequence comparison in FIG. 5) or the rat (rattus norvegicus, see human sequence comparison FIG. 6).

In preferred embodiments, variants of human transforming growth factor-beta (TGF-β) type III receptor are polypeptides with greater than 50, 60, 70, 80, 90, 95, or 99% identity or similarity to (SEQ ID NO: 1)

MTSHYVIAIFALMSFCLATAGPEPGALCELSPVSASHPVQALMESFTVLSGCASRGTTGL PQEVHVLNLRTAGQGPGQLQREVTLHLNPISSVHIFIFIKSVVFLLNSPHPLVWHLKTERL ATGVSRLFLVSEGSVVQFSSANFSLTAETEERNFPHGNEHLLNWARKEYGAVTSFTELKI ARNIYIKVGEDQVFPPKCNIGKNFLSLNYLAEYLQPKAAEGCVMSSQPQNEEVHIIELITP NSNPYSAFQVDITIDIRPSQEDLEVVKNLILILKCKKSVNWVIKSFDVKGSLKIIAPNSIGF GKESERSMTMTKSIRDDIPSTQGNLVKWALDNGYSPITSYTMAPVANRFHLRLENNAEE MGDEEVHTIPPELRILLDPGALPALQNPPIRGGEGQNGGLPFPFPDISRRVWNEEGEDGLP RPKDPVIPSIQLFPGLREPEEVQGSVDIALSVKCDNEKMIVAVEKDSFQASGYSGMDVTL LDPTCKAKMNGTHFVLESPLNGCGTRPRWSALDGVVYYNSIVIQVPALGDSSGWPDGY EDLESGDNGFPGDMDEGDASLFTRPEIVVFNCSLQQVRNPSSFQEQPHGNITFNMELYNT DLFLVPSQGVFSVPENGHVYVEVSVTKAEQELGFAIQTCFISPYSNPDRMSHYTIIENICP KDESVKFYSPKRVHFPIPQADMDKKRFSFVFKPVFNTSLLFLQCELTLCTKMEKHPQKLP KCVPPDEACTSLDASIIWAMMQNKKTFTKPLAVIHHEAESKEKGPSMKEPNPISPPIFHG LDTLTVMGIAFAAFVIGALLTGALWYTYSHTGETAGRQQVPTSPPASENSSAAHSIGSTQ STPCSSSSTA, or polypeptides with greater than 50, 60, 70, 80, 90, 95, or 99% identity or similarity to SEQ ID NO: 4 (N-terminal cellular domain)
TSHYVIAIFALMSFCLATAGPEPGALCELSPVSASHPVQALMESFTVLSGCASRGTTGLP QEVHVLNLRTAGQGPGQLQREVTLHLNPISSVHIFIRKSVVFLLNSPHPLVWHLKTERLA TGVSRLFLVSEGSVVQFSSANFSLTAETEERNFPHGNEHLLNWARKEYGAVTSFTELKIA RNIYIKVGEDQVFPPKCNIGKNFLSLNYLAEYLQPKAAEGCVMSSQPQNEEVHIIELITPN SNPYSAFQVDITIDIRPSQEDLEVVKNLILILKCKKSVNWVIKSFDVKGSLKIIAPNSIGFG KESERSMTMTKSIRDDIPSTQGNLVKWALDNGYSPITSYTMAPVANRFHLRLENNAEEM GDEEVHTIPPELRILLDPGALPALQNPPIRGGEGQNGGLPFPFPDISRRVWNEEGEDGLPR PKDPVIPSIQLFPGLREPEEVQGSVDIALSV, or polypeptides with greater than 50, 60, 70, 80, 90, 95, or 99% identity or similarity to SEQ ID NO: 5 (N-terminal cellular domain and the Zona pellucida (ZP) domain)
TSHYVIAIFALMSFCLATAGPEPGALCELSPVSASHPVQALMESFTVLSGCASRGTTGLP QEVHVLNLRTAGQGPGQLQREVTLHLNPISSVHIFIRKSVVFLLNSPHPLVWHLKTERLA TGVSRLFLVSEGSVVQFSSANFSLTAETEERNFPHGNEHLLNWARKEYGAVTSFTELKIA RNIYIKVGEDQVFPPKCNIGKNFLSLNYLAEYLQPKAAEGCVMSSQPQNEEVHIIELITPN SNPYSAFQVDITIDIRPSQEDLEVVKNLILILKCKKSVNWVIKSFDVKGSLKIIAPNSIGFG KESERSMTMTKSIRDDIPSTQGNLVKWALDNGYSPITSYTMAPVANRFHLRLENNAEEM GDEEVHTIPPELRILLDPGALPALQNPPIRGGEGQNGGLPFPFPDISRRVWNEEGEDGLPR PKDPVIPSIQLFPGLREPEEVQGSVDIALSVKCDNEKMIVAVEKDSFQASGYSGMDVTLL DPTCKAKMNGTHFVLESPLNGCGTRPRWSALDGVVYYNSIVIQVPALGDSSGWPDGYE DLESGDNGFPGDMDEGDASLFTRPEIVVFNCSLQQVRNPSSFQEQPHGNITFNMELYNT DLFLVPSQGVFSVPENGHVYVEVSVTKAEQELGFAIQTCFISPYSNPDRMSHYTIIENICP KDESVKFYSPKRVHFPIPQADMDKKRFSFVFKPVFNTSLLFLQCELTLCTKMEKHPQKLP KCVP.

In certain embodiments, the TβRIII variant comprises one or two amino acid substitutions. In certain embodiments, the TβRIII variant comprises one or two conserved amino acid substitutions. In certain embodiments, the TβRIII variant comprises one or two amino acid insertions. In certain embodiments, the TβRIII variant comprises one or two amino acid deletions. In certain embodiments, the TβRIII variant comprises three or four amino acid substitutions. In certain embodiments, the TβRIII variant comprises three or four conserved amino acid substitutions. In certain embodiments, the TβRIII variant comprises three or four amino acid insertions. In certain embodiments, the TβRIII variant comprises three or four amino acid deletions. In certain embodiments, the TβRIII variant comprises five or six amino acid substitutions. In certain embodiments, the TβRIII variant comprises five or six conserved amino acid substitutions. In certain embodiments, the TβRIII variant comprises five or six amino acid insertions. In certain embodiments, the TβRIII variant comprises five or six amino acid deletions. In certain embodiments, the TβRIII variant comprises at least one non-naturally occurring mutation. In certain embodiments, the TβRIII variant comprises at least one N or C terminal amino acid that is not present in a human sequence such that the entire sequence is not naturally occurring. In certain embodiments, the TβRIII variant is truncated human sequence such that the truncated sequence does not naturally occurring. In certain embodiments, the TβRIII variant comprises at least one amino acid insertion or deletion that is not present in a human sequence such that the entire sequence is not naturally occurring. In certain embodiments, TβRIII or variant are covalently bonded to a matrix material or hydrogel peptide or other synthetic molecule such that TβRIII is structurally modified when compared to naturally occurring TβRIII.

In certain embodiments, TβRIII or variant of are a recombinant TβRIII, a TβRIII homolog, ortholog, fragment, or mutant. Polypeptides comprising a TβRIII sequence or active variant or fragment include chimeric proteins. In certain embodiments, the polypeptide is a TβRIII or variant, fusion protein, e.g., TβRIII conjugated to antibody or antibody fragment.

A nucleic acid sequence encoding TβRIII or variant can readily be obtained in a variety of ways, including, without limitation, chemical synthesis, cDNA or genomic library screening, expression library screening, and/or PCR amplification of cDNA. These methods and others useful for isolating such nucleic acid sequences are set forth, for example, by Sambrook et al. (Molecular Cloning: A Laboratory Manual, Cold Spring Harbor Laboratory Press, Cold Spring Harbor, N.Y., 1989), by Ausubel et al., eds (Current Protocols in Molecular Biology, Current Protocols Press, 1994), and by Berger and Kimmel (Methods in Enzymology: Guide to Molecular Cloning Techniques, vol. 152, Academic Press, Inc., San Diego, Calif, 1987).

Chemical synthesis of a nucleic acid sequence which encodes a polypeptide can also be accomplished using methods well known in the art, such as those set forth by Engels et al. (Angew. Chem. Intl. Ed., 28:716-734, 1989). These methods include, inter alia, the phosphotriester, phosphoramidite and H-phosphonate methods of nucleic acid sequence synthesis. The nucleic acid sequence encoding the TβRIII will be several hundred base pairs (bp) or nucleotides in length. Large nucleic acid sequences, for example those larger than about 100 nucleotides in length, can be synthesized as several fragments. The fragments can then be ligated together to form a nucleic acid sequence encoding TβRIII. A preferred method is polymer-supported synthesis using standard phosphoramidite chemistry.

Alternatively, a suitable nucleic acid sequence may be obtained by screening an appropriate cDNA library (i.e., a library prepared from one or more tissue source(s) believed to express the protein) or a genomic library (a library prepared from total genomic DNA). The source of the cDNA library is typically a tissue from any species that is believed to express TβRIII in reasonable quantities. The source of the genomic library may be any tissue or tissues from any mammalian or other species believed to harbor a gene encoding TβRIII or a TβRIII homologue. The library can be screened for the presence of the TβRIII cDNA/gene using one or more nucleic acid probes (oligonucleotides, cDNA or genomic DNA fragments that possess an acceptable level of homology to the TβRIII or TβRIII homologue cDNA or gene to be cloned) that will hybridize selectively with TβRIII or TβRIII homologue cDNA(s) or gene(s) present in the library. The probes typically used for such library screening usually encode a small region of the TβRIII DNA sequence from the same or a similar species as the species from which the library was prepared. Alternatively, the probes may be degenerate.

The cDNA or genomic DNA encoding a polypeptide is inserted into a vector for further cloning (amplification of the DNA) or for expression. Suitable vectors are commercially available, or the vector may be specially constructed. The selection or construction of the appropriate vector will depend on 1) whether it is to be used for DNA amplification or for DNA expression, 2) the size of the DNA to be inserted into the vector, and 3) the host cell (e.g., mammalian, insect, yeast, fungal, plant or bacterial cells) to be transformed with the vector. Each vector contains various components depending on its function (amplification of DNA or expression of DNA) and its compatibility with the intended host cell. The vector components generally include, but are not limited to, one or more of the following: a signal sequence, an origin of replication, one or more selection or marker genes, enhancer elements, promoters, a transcription termination sequence, and the like. These components may be obtained from natural sources or synthesized by known procedures. The vectors of the present invention involve a nucleic acid sequence which encodes the polypeptide of interest operatively linked to one or more of the following expression control or regulatory sequences capable of directing, controlling or otherwise effecting the expression of the polypeptide by a selected host cell.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and occasionally 3′ untranslated regions of eukaryotic DNAs or cDNAs. These regions contain nucleotide segments transcribed as polyadenylated fragments in the untranslated portion of the mRNA encoding polypeptide.

The construction of suitable vectors containing one or more of the above-listed components together with the desired polypeptide coding sequence is accomplished by standard ligation techniques. Isolated plasmids or DNA fragments are cleaved, tailored, and re-ligated in the desired order to generate the plasmids required. To confirm that the correct sequences have been constructed, the ligation mixtures may be used to transform E. coli, and successful transformants may be selected by known techniques, such as ampicillin or tetracycline resistance as described above. Plasmids from the transformants are then prepared, analyzed by restriction endonuclease digestion, and/or sequenced to confirm the presence of the desired construct.

Host cells (e.g., bacterial, mammalian, insect, yeast, or plant cells) transformed with nucleic acid sequences for use in expressing a recombinant polypeptides are also provided by the present disclosure. The transformed host cell is cultured under appropriate conditions permitting the expression of the nucleic acid sequence. The selection of suitable host cells and methods for transformation, culture, amplification, screening and product production and purification are well known in the art. See for example, Gething and Sambrook, Nature 293: 620-625 (1981), or alternatively, Kaufman et al., Mol. Cell. Biol., 5 (7): 1750-1759 (1985) or Howley et al., U.S. Pat. No. 4,419,446.

Transformed cells used to produce polypeptides of the present invention are cultured in suitable media. The media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamicin), trace elements (defined as inorganic compounds usually present at final concentrations in the micromolar range), and glucose or other energy source. Other supplements may also be included, at appropriate concentrations, as will be appreciated by those skilled in the art. Suitable culture conditions, such as temperature, pH, and the like, are also well known to those skilled in the art for use with the selected host cells.

Chemically modified derivatives of TβRIII or TβRIII variants may be prepared by one skilled in the art given the disclosures herein. The chemical moieties most suitable for derivatization of polypeptide include water soluble polymers. A water soluble polymer is desirable because the protein to which it is attached does not precipitate in an aqueous environment, such as a physiological environment. Preferably, the polymer will be pharmaceutically acceptable for the preparation of a therapeutic product or composition. One skilled in the art will be able to select the desired polymer based on such considerations as whether the polymer/protein conjugate will be used therapeutically, and if so, the desired dosage, circulation time, resistance to proteolysis, and other considerations. The effectiveness of the derivatization may be ascertained by administering the derivative, in the desired form (i.e., by osmotic pump, or, more preferably, by injection or infusion, or further formulated for oral, pulmonary or other delivery routes), and determining its effectiveness.

Suitable water soluble polymers include, but are not limited to, polyethylene glycol (PEG), copolymers of ethylene glycol/propylene glycol, monomethoxy-polyethylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, propylene glycol homopolymers, polypropylene oxide/ethylene oxide co-polymers, polyoxyethylated polyols (e.g., glycerol), polyethylene glycol propionaldehyde, and mixtures thereof. As used herein, polyethylene glycol is meant to encompass any of the forms of PEG that have been used to derivatize other proteins, such as mono-(C₁-C₁₀) alkoxy- or aryloxy-polyethylene glycol. Polyethylene glycol propionaldehyde may have advantages in manufacturing due to its stability in water. The polymer may be of any molecular weight, and may be branched or unbranched.

The present disclosure particularly relates to TβRIII or variant linked to at least one PEG molecule. In another aspect, the present disclosure relates to TβRIII attached to at least one PEG molecule via an acyl or alkyl linkage.

Pegylation may be carried out by any of the pegylation reactions known in the art. See, for example: Focus on Growth Factors 3(2): 4-10 (1992); EP 0 154 316; EP 0 401384; and Malik et al., Exp. Hematol. 20: 1028-1035 (1992) (reporting pegylation of GM-CSF using tresyl chloride). Preferably, the pegylation is carried out via an acylation reaction or an alkylation reaction with a reactive water soluble polymer. These preferred means for derivatization are discussed in greater detail, below. For the acylation reactions, the polymer(s) selected preferably have a single reactive ester group. For the reductive alkylation reactions, the polymer(s) selected preferably have a single reactive aldehyde group. In addition, the selected polymer may be modified to have a single reactive group, such as an active ester for acylation or an aldehyde for alkylation, so that the degree of polymerization may be controlled. Generally, the water soluble polymer will not be selected from naturally-occurring glycosyl residues since these are usually made more conveniently by mammalian recombinant expression systems.

### Growth Factors

In some embodiments, the disclosure relates to the combined use of TβRIII or variants and growth factor(s) in bone growth applications. Typically, the growth factor is a bone morphogenetic proteins (BMPs), including but not limited to, BMP-1, BMP-2, BMP-2A, BMP-2B, BMP-3, BMP-3b, BMP-4, BMP-5, BMP-6, BMP-7 (0P-1), BMP-8, BMP-8b, BMP-9, BMP-10, BMP-11, BMP-12, BMP-13, BMP-14, and BMP-15. BMPs act through specific transmembrane receptors located on cell surface of the target cells.

Non-limiting examples of additional suitable growth factors include osteogenin, insulin-like growth factor (IGF)-1, IGF-II, TGF-betal, TGF-beta2, TGF-beta3, TGF-beta4, TGF-beta5, osteoinductive factor (OIF), basic fibroblast growth factor (bFGF), acidic fibroblast growth factor (aFGF), platelet-derived growth factor (PDGF), vascular endothelial growth factor (VEGF), growth hormone (GH), growth and differentiation factors (GDF)-5 through 9, and osteogenic protein-1 (OP-1). The growth factors may be isolated from synthetic methods, recombinant sources or may be purified from a biological sample. Preferably the growth factors are obtained from a recombinant technology and for clarity certain embodiments include rhBMP-2, rhBMP-4, rhBMP-6, rhBMP-7, and rhGDF-5, as disclosed, for example, in the U.S. Pat. Nos. 4,877,864; 5,013,649; 5,661,007; 5,688,678; 6,177,406; 6,432,919; 6,534,268, and 6,858,431; and in Wozney, J. M., et al. (1988) Science, 242(4885):1528-1534, all hereby incorporated by reference.

In a typical embodiment, a graft composition comprises a matrix, TβRIII, and BMP-2. In one embodiment, the matrix contains an effective amount of a BMP-2 protein, an rhBMP-2 protein, functional fragments thereof, or combinations thereof. Although a graft matrix may be loaded during manufacturing, it is typically loaded just prior to implantation.

The transcription of human BMP-2 is 396 amino acids in length, localized to chromosome 20p12. BMP-2 belongs to the transforming growth factor-beta (TGF-beta) superfamily. The human amino acid sequence BMP-2 is SEQ ID NO: 6 shown below. Amino acids 38-268 are the TGF-beta propeptide domain, and 291-396 are the TGF-beta family N-terminal domain. Amino acids 283-396 are the mature peptide. The mature form of BMP-2 contains four potential N-linked glycosylation sites per polypeptide chain, and four potential disulfide bridges. (SEQ ID NO: 6) MVAGTRCLLALLLPQVLLGGAAGLVPELGRRKFAAASSGRPSSQPSDEVLSEFELRLLS NIFGLKQRPTPSRDAVVPPYMLDLYRRHSGQPGSPAPDHRLERAASRANTVRSFHHEESL EELPETSGKTTRRFFFNLSSIPTEEFITSAELQVFREQMQDALGNNSSFHHRINIYEIIKPAT ANSKFPVTRLLDTRLVNQNASRWESFDVTPAVIVIRWTAQGHANHGFVVEVAHLEEKQG VSKRHVRISRSLHQDEHSWSQIRPLLVTFGHDGKGHPLHKREKRQAKHKQRKRLKSSC KRHPLYVDFSDVGWNDWIVAPPGYHAFYCHGECPFPLADHLNSTNHAIVQTLVNSVNS KIPKACCVPTELSAISMLYLDENEKVVLKNYQDMVVEGCGCR.

### EXAMPLES

### In Vitro Osteoblast Commitment Using Full Length TβRIII

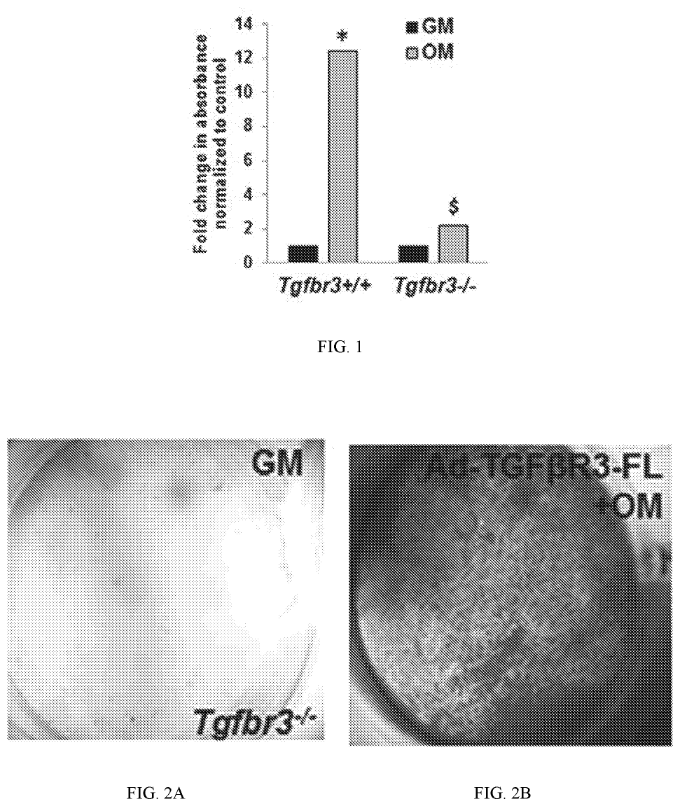

Culture of the palate mesenchymal cells demonstrated that they were unable to commit to osteoblast fate and mineralize in osteogenic media (FIG. 1). The cell fate of the TβRIII−/− mesenchymal cells was rescued with adenoviral vector expressing TβRIII-FL, and the cells were able to undergo osteoblast commitment and mineralize (FIG. 2B). Additional analysis of related genes revealed that multiple other genes involved in cell fate commitment and intracellular cytostructure were down regulated including GIPC, RhoA, Rac1, Cdc42. These data indicate that TβRIII is helpful for intramembranous ossification.

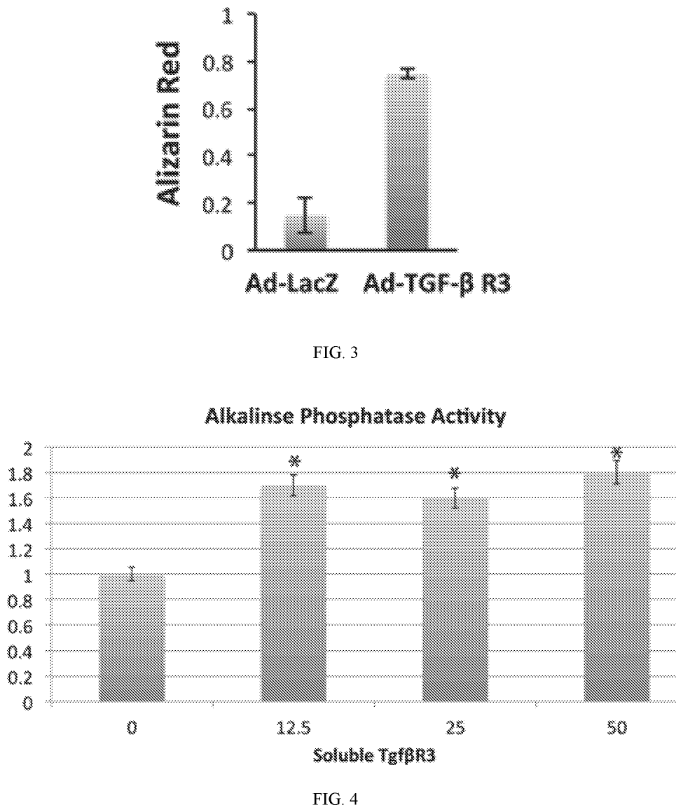

The use of human embryonic palate mesenchyme (HEPM), a similar cell line to the palate mesenchymal studies, was investigated. HEPM cells undergo osteoblast commitment and mineralize when using and adenoviral vector TβRIII-FL for overexpression. The control adenoviral-LacZ vector cells did not (FIG. 3).

The addition of soluble TβRIII to the osteogenic media indicated an increased osteoblast commitment using an alkaline phosphatase assay (FIG. 4). Increased transcription of genes involved in osteogenic commitment were also observed. Infection of the HEPM cells with the adenoviral vector at MOI of 100 demonstrated strong GFP signaling. The adenoviral infection did not alter cellular proliferation or apoptosis. The expression of TβRIII mRNA was strongly detected with 4-fold increase in the adenoviral TβRIII -FL infected cells compared to controls. Infection of the HEPM cells with adenoviral TβRIII -FL significantly increased osteoblast commitment measured by alkaline phosphatase and alizarin red compared to control cells in osteogenic media.

### Soluble TβRIII and Mesenchymal Cells Overexpressing TβRIII Using PEG-MAL Hydrogel Composition:

PEG-MAL hydrogels (4% wt/v) may be synthesized by reacting PEG-MAL [Four-arm maleimide end-functionalized PEG macromer (PEG-MAL) (20kDA, Laysan Bio)] with recombinant TβRIII (R&D Systems Gly21-Asp781, or overexpressed TβRIII shedded receptor) or albumin with a dithiol protease-cleavable peptide (VPM) cross-linker GCRDVPMSMRGGDRCG (SEQ ID NO: 7) at a volume ratio of 2:1:1. See Phelps et al. Adv Mater. 2012, 24(1): 64-2.

The concentration of used for the synthesis of each hydrogel may be calculated to match the number of cysteine residues on the peptide cross-linker with the number of free (unreacted) maleimide groups remaining in the adhesive peptide-functionalized PEG-maleimide solution. The mixture of peptide-functionalized PEG-maleimide, soluble TβRIII and VPM containing cross-linker may be incubated to allow for cross-linking before adding PBS to the hydrogels.

### In Vivo Model of Maxillary Bone Grafting

BMP2 causes bone formation that can be uncontrolled and lead to heterotopic ossification (bone in the wrong spot). BMP2 is not used in pediatric patients. To assess the ability of TβRIII to induce bone formation in vivo a calvaria bone defect was used. A circular parietal defect of 3 mm was created using a drill. The calvaria was left empty or filed with soluble TβRIII in hydrogel (PEG-MAL scaffold). After 12 weeks, the bone deposition was evaluated by 3D X-Ray Micro-CT and immunohistochemistry. The micro-CT images showed that soluble TβRIII was able to induce bone formation compare to the empty defect with hydrogel. The immunohistochemistry stain showed the complete recovery of around the edge of the defect in the mouse treated with soluble TβRIII in hydrogel. Bone volume increased significantly more in the mice that were treated with 5 uM of BMP2. TβRIII promotes local bone progenitors to form to a lesser extent. A benefit of TβRIII is that it has a milder osteogenic effect preventing heterotopic ossification. TβRIII is an alternative that can be delivery locally to grow bone in a more effective manner

1. A bone graft composition comprising a transforming growth factor-beta (TGF-β) type III receptor.

2. The bone graft composition of claim 1, wherein the transforming growth factor-beta (TGF-β) type III receptor comprises SEQ ID NO: 4.

3. The bone graft composition of claim 1, wherein transforming growth factor-beta (TGF-β) type III receptor is overexpressed on a cell.

4. The bone graft composition of claim 1, comprising polyethylene glycol.

5. The bone graft composition of claim 1, wherein said graft comprises collagen or a hydrogel.

6. A kit comprising a transforming growth factor-beta (TGF-β) type III receptor and a graft composition.

7. The kit of claim 6 wherein the graft composition comprises polyethylene glycol, collagen, or hydrogel matrix.

8. A method of forming bone, comprising implanting a graft composition comprising transforming growth factor-beta (TGF-β) type III receptor in a subject under conditions such that bone forms in the graft composition.

9. The method of claim 8, wherein the subject has a void in the bony structure wherein the graft composition is implanted in the void.

10. The method of claim 9, wherein the void is in a bone of the maxilla, face, or cranium.

11. The method of claim 10, wherein said void is a result of an orofacial cleft, cleft palate, cleft lip or surgical removal of bone.

12. A method of treating a bone fracture comprising administering a pharmaceutical composition comprising transforming growth factor-beta (TGF-β) type III receptor to a subject at risk for, exhibiting symptoms of, or diagnosed with a bone fracture.

13. The method of claim 12, wherein the administration is localized by percutaneous injection.

14. A method of preventing or treating a bone degenerative disease comprising administering a pharmaceutical composition comprising transforming growth factor-beta (TGF-β) type III receptor to a subject at risk for, exhibiting symptoms of, or diagnosed with a disease.

15. The method of claim 14, wherein the disease is osteoporosis, osteitis deformans, bone metastasis, multiple myeloma, primary hyperparathyroidism, or osteogenesis imperfecta.


SEQUENCE LISTING (11 sequences)

SEQ ID NO: 1 - protein, 841 aa, Artificial
  Synthetic construct
MTSHYVIAIFALMSFCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQ

SEQ ID NO: 2 - protein, 832 aa, Artificial
  Synthetic construct
VTSHHMVPVFVLMSACLATAGPEPSTRCELSPISASHPVQALMESFTVLSGCASRGTTGLPREVHILNLRSTDQGLGQPQREVTLHLNPIASVHTHHKPVVFLLNSPQPLVWHVKTERLAAGVPRLFLVSEGSVVQFSSGNFSLTAETEERSFPQENEHLLHWAQKEYGAVTSFTELKIARNIYIKVGEDQVFPPTCNIGKNFLSLNYLAEYLQPKAAEGCVLASQPHEKEVHIIELISPNSNPYSTFQVDIIIDIRPAREDPEVVKNLVLILKCKKSVNWVIKSFDVKGNLKVIAPDSIGFGKESERSMTVTKLVRNDIPSTQENLMKWALDNGYSPVTSYTIAPVANRFHLRLENNEEMRDEEVHTIPPELRILLGPDHLPALDSPSFQGEIPNGGFPFPFPDIPRRGWKEGEDRIPRPKEPIIPRVQLLPDHREPEEVQGGVNIALSVKCDNEKMVVAVDKDSFQTNGYSGMELTLLDPSCKAKMNGTHFVLESPLNGCGTRHRRSAPDGVVYYNSIVVQAPSPGDSSGWPDGYEDLESGDNGFPGDTDEGETAPLSRAGVVVFNCSLRQLRSPSGFQDQLDGNATFNMELYNTDLFLVPSPGVFSVAENEHVYVEVSVTKADQDLGFAIQTCFISPYSNPDRMSDYTIIENICPKDDSVKFYSSKRVHFPIPHAEVDKKRFSFVFKSVFNTSLLFLHCELTLCSRKKGSQKLPKCVTPDDACTSLDATMIWTMMQNKKTFTKPLAVVLQVDYKENVPNMKESSPVPPPPQIFHGLDTLTVMGIAFAAFVIGALLTGALWYIYSHTGETARRQQVPTSPPASENSSAAH

SEQ ID NO: 3 - protein, 775 aa, Artificial
  Synthetic construct
LPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPDHPPALDNPLFPGEGSPNGGLPFPFPDIPRRGWKEGEDRIPRPKQPIVPSVQLLPDHREPEEVQGGVDIALSVKCDHEKMVVAVDKDSFQTNGYSGMELTLLDPSCKAKMNGTHFVLESPLNGCGTRHRRSTPDGVVYYNSIVVQAPSPGDSSGWPDGYEDLESGDNGFPGDGDEGETAPLSRAGVVVFNCSLRQLRNPSGFQGQLDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVDYKENVPSTKDSSPIPPPPPQIFHGLDTLTVMGIAFAAFVIGALLTGALWYIYSHTGETARRQQVPTSPPASENSSAAH

SEQ ID NO: 4 - protein, 453 aa, Artificial
  Synthetic construct
TSHYVIAIFALMSFCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSV

SEQ ID NO: 5 - protein, 724 aa, Artificial
  Synthetic construct
TSHYVIAIFALMSFCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVP

SEQ ID NO: 6 - protein, 396 aa, Artificial
  Synthetic construct
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 7 - protein, 16 aa, Artificial
  Synthetic construct
GCRDVPMSMRGGDRCG

SEQ ID NO: 8 - protein, 6 aa, Artificial
  Synthetic construct
GGGGGG

SEQ ID NO: 9 - protein, 5 aa, Artificial
  Synthetic construct
GGGGT

SEQ ID NO: 10 - protein, 6 aa, Artificial
  Synthetic construct
GGGPPP

SEQ ID NO: 11 - protein, 7 aa, Artificial
  Synthetic construct
GGGAPPP